(19) United States
(12) Patent Application Publication
(10) Pub. No.: US 2022/0273244 A1
(43) Pub. Date: Sep. 1, 2022

(54) PHYSIOLOGICAL SIGNAL RECOGNITION APPARATUS AND PHYSIOLOGICAL SIGNAL RECOGNITION METHOD

(71) Applicant: Industrial Technology Research Institute, Hsinchu (TW)

(72) Inventors: Heng-Yin Chen, Hsinchu County (TW); Yun-Yi Huang, Pingtung County (TW); Shuen-Yu Yu, New Taipei City (TW)

(73) Assignee: Industrial Technology Research Institute, Hsinchu (TW)

(21) Appl. No.: 17/223,012

(22) Filed: Apr. 6, 2021

(30) Foreign Application Priority Data
Feb. 26, 2021 (TW) 110106860

(51) Int. Cl. A61B 5/00; A61B 5/22; A61B 5/296; A61B 5/397; G16H 20/30; G16H 40/67

(52) U.S. Cl. CPC A61B 5/7207; A61B 5/224; A61B 5/296; A61B 5/397; G16H 20/30; G16H 40/67

(57) ABSTRACT

A physiological signal recognition apparatus and a physiological signal recognition method are provided. A root mean square algorithm is executed on a physiological signal to obtain a noise threshold, and the physiological signal is adjusted based on the noise threshold to obtain an adjusted signal. Then, a muscle strength starting point in the adjusted signal is detected.

### CROSS-REFERENCE TO RELATED APPLICATION

This application claims the priority benefit of Taiwan application serial no. 110106860, filed on Feb. 26, 2021. The entirety of the above-mentioned patent application is hereby incorporated by reference herein and made a part of this specification.

### BACKGROUND

### Technical Field

The disclosure relates to a signal processing mechanism, and also relates to a physiological signal recognition apparatus and a physiological signal recognition method.

### Description of Related Art

Modern people increasingly rely on wearable smart apparatuses to sense physiological signals, so as to always pay attention to physical conditions and effectively manage their health. Nowadays, most people generally pay attention to their own health, and also spare time to do some exercise apart from work. It is a very convenient choice whether to exercise at home or go to the gym. Based on the high correlation between electromyography (EMG) signals and motion, the analysis of the EMG signals has become a hot research topic and is widely applied in many fields. The EMG signal may be used to determine the degree of muscle fatigue. The time domain analysis may monitor possible conditions and peripheral fatigue, and the frequency domain analysis may understand the excitation rate of a motor unit. At present, there are many indicators in the time domain and frequency domain analyses that may be used as references for medical applications. However, the EMG signals may be distorted and difficult to be interpreted due to large background noise and other muscle and electrode distance noise variations.

### SUMMARY

The disclosure provides a physiological signal recognition apparatus, which includes a physiological signal sensor, sensing a physiological signal; and a processor, coupled to the physiological signal sensor and configured to: execute a root mean square (RMS) algorithm on the physiological signal to obtain a noise threshold; adjust the physiological signal based on the noise threshold to obtain an adjusted signal; and detect a muscle strength starting point in the adjusted signal.

The physiological signal recognition method of the disclosure includes the following steps. A physiological signal is converted into an initial frequency domain signal. A noise variation is calculated based on a compensation value obtained by a compensation element. A noise frequency corresponding to the noise variation is found from a database. The noise frequency in the initial frequency domain signal is removed to obtain a corrected frequency domain signal. The corrected frequency domain signal is converted into a time domain signal. The time domain signal is recorded as a corrected physiological signal.

The physiological signal recognition method of the disclosure includes the following steps. A physiological signal is converted into an initial frequency domain signal. The initial frequency domain signal is compared with a standard signal to obtain a noise frequency. The noise frequency in the initial frequency domain signal is removed to obtain a corrected frequency domain signal. The corrected frequency domain signal is converted into a time domain signal. The time domain signal is recorded as a corrected physiological signal.

Several exemplary embodiments accompanied with figures are described in detail below to further describe the disclosure in details.

### BRIEF DESCRIPTION OF THE DRAWINGS

The accompanying drawings are included to provide further understanding, and are incorporated in and constitute a part of this specification. The drawings illustrate exemplary embodiments and, together with the description, serve to explain the principles of the disclosure.

FIG. 1 is a block diagram of a physiological signal recognition apparatus according to an embodiment of the disclosure.

FIG. 2 is a block diagram of a system module according to an embodiment of the disclosure.

FIG. 3A and FIG. 3B are schematic diagrams of physiological signals according to an embodiment of the disclosure.

FIG. 4 is a block diagram of a physiological signal recognition apparatus according to an embodiment of the disclosure.

FIG. 5 is a flowchart of a physiological signal recognition method according to an embodiment of the disclosure.

FIG. 6 is a schematic diagram of sensing electrodes according to an embodiment of the disclosure.

FIG. 7 is a block diagram of a physiological signal recognition apparatus according to an embodiment of the disclosure.

FIG. 8 is a flowchart of a physiological signal recognition method according to an embodiment of the disclosure.

FIG. 9 is a block diagram of a system module according to an embodiment of the disclosure.

FIG. 10 is a block diagram of a system module according to an embodiment of the disclosure.

### DETAILED DESCRIPTION OF DISCLOSED EMBODIMENTS

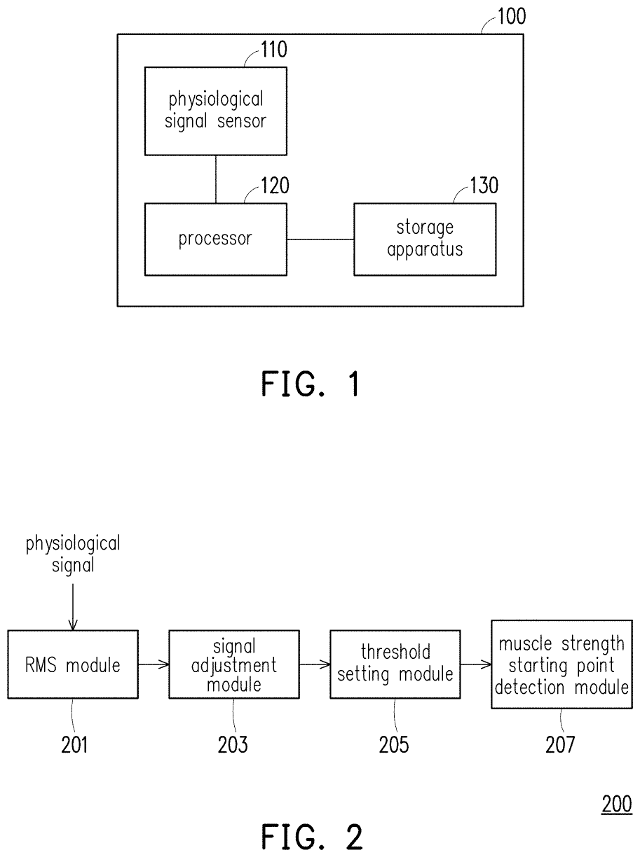

FIG. 1 is a block diagram of a physiological signal recognition apparatus according to an embodiment of the disclosure. Please refer to FIG. 1. A physiological signal recognition apparatus 100 includes a physiological signal sensor 110, a processor 120, and a storage apparatus 130. The processor 120 is coupled to the physiological signal sensor 110 and the storage apparatus 130.

The physiological signal sensor 110 is configured to detect a physiological signal. The physiological signal is, for example, an electromyography (EMG) signal. The processor 120 is, for example, a central processing unit (CPU), a physics processing unit (PPU), a programmable microprocessor, an embedded control chip, a digital signal processor (DSP), an application specific integrated circuits (ASIC), or other similar apparatuses.

The storage apparatus 130 is, for example, any type of fixed or removable random-access memory, read-only memory, flash memory, secure digital card, hard disk, other similar apparatuses, or a combination of these apparatuses. Multiple code snippets are stored in the storage apparatus 130. The code snippets are executed by the processor 120 after being installed to execute a physiological signal recognition method. The physiological signal recognition method includes: executing a root mean square (RMS) algorithm on a physiological signal to obtain a noise threshold, adjusting the physiological signal based on the noise threshold to obtain an adjusted signal, and detecting a muscle strength starting point in the adjusted signal.

The code snippets may be composed into a system module, as shown in FIG. 2. FIG. 2 is a block diagram of a system module according to an embodiment of the disclosure. In FIG. 2, a system module 200 includes an RMS module 201, a signal adjustment module 203, a threshold setting module 205, and a muscle strength starting point detection module 207. The physiological signal is transmitted to the RMS module 201, and the RMS module 201 executes the RMS algorithm on the physiological signal to obtain the noise threshold. Then, the signal adjustment module 203 adjusts the physiological signal based on the noise threshold. For example, an amplitude in the physiological signal that is less than the noise threshold is multiplied by a first weight value and an amplitude in the physiological signal that is greater than or equal to the noise threshold is multiplied by a second weight value to obtain the adjusted signal.

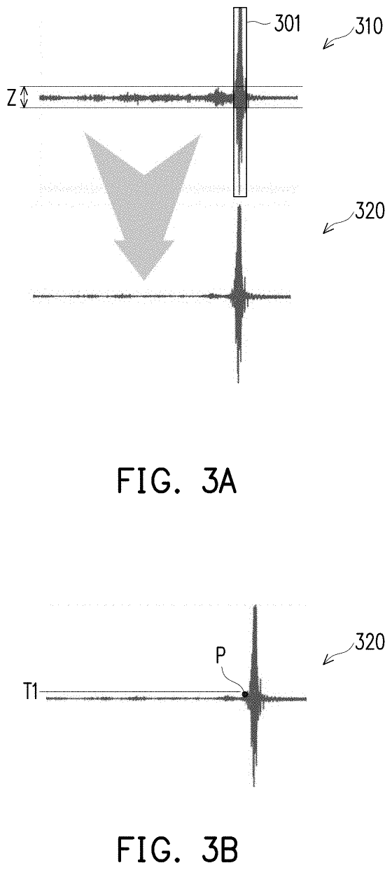

FIG. 3A and FIG. 3B are schematic diagrams of physiological signals according to an embodiment of the disclosure. In FIG. 3A, an amplitude in a physiological signal 310 that is less than a noise threshold Z is multiplied by the first weight value and an amplitude in the physiological signal 310 that is greater than or equal to the noise threshold Z (that is, the amplitude in a main frequency region 301) is multiplied by the second weight value to obtain an adjusted signal 320. Here, the first weight value is, for example, 0.01 and the second weight value is, for example, 1. That is, the amplitude less than the noise threshold Z is regarded as noise, so the amplitude regarded as noise is multiplied by 0.01 to reduce the influence thereof. On the other hand, the amplitude greater than or equal to the noise threshold Z is regarded as the main frequency, so the amplitude regarded as a muscle strength signal is multiplied by 1 to maintain the signal strength thereof without reducing the amplitude of the main frequency. In addition, in other embodiments, the first weight value may also be any other value, which is not limited thereto.

After the adjusted signal 320 is obtained, as shown in FIG. 3B, the threshold setting module 205 sets a starting signal threshold T1 based on the adjusted signal. Here, the threshold setting module 205 may set the starting signal threshold T1 according to an action speed at which the muscle completes a specific action. When the action speed is fast, the starting signal threshold T1 is set to high; and when the action speed is slow, the starting signal threshold T1 is set to low. For example, the processor 120 judges the speed of the action according to the duration of a waveform in the physiological signal, which is also the frequency of a signal waveform oscillation. The smaller the frequency, the slower the action. Conversely, the larger the frequency, the faster the action. Therefore, the action speed may be detected according to the magnitude of the frequency. The description here is implementable. Accordingly, the processor 120 may judge the action speed according to the waveform of the physiological signal every time the user wears the physiological signal recognition apparatus 100 to execute a specific action. Accordingly, the starting signal threshold T1 is adjusted based on the action speed, thereby improving the recognition rate of the muscle strength starting point. After the starting signal threshold T1 is obtained, the muscle strength starting point detection module 207 detects a muscle strength starting point P in the adjusted signal 320 based on the starting signal threshold T1. For example, when a point where the signal suddenly continues to be greater than the starting signal threshold T1 is detected, the point is set as the muscle strength starting point P.

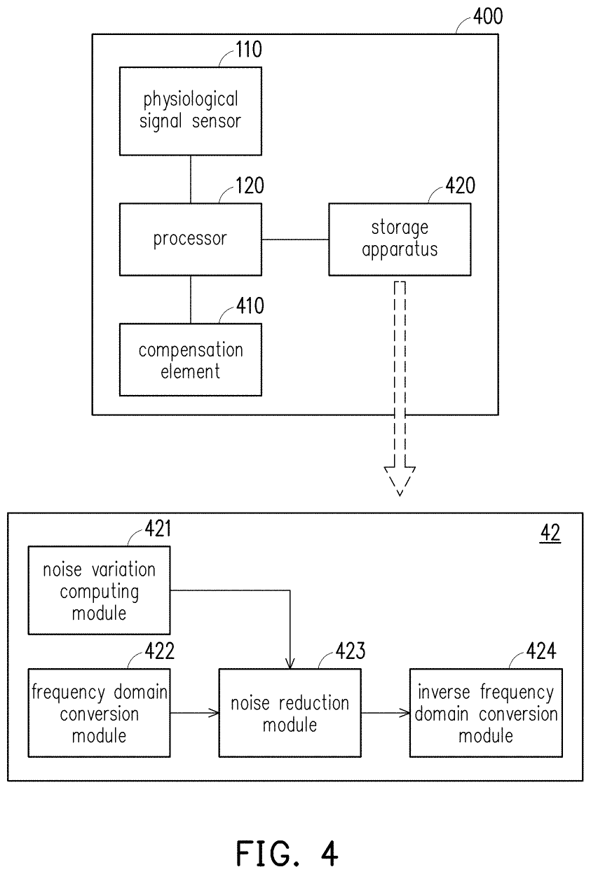

FIG. 4 is a block diagram of a physiological signal recognition apparatus according to an embodiment of the disclosure. Please refer to FIG. 4. A physiological signal recognition apparatus 400 includes a physiological signal sensor 110, a processor 120, a compensation element 410, and a storage apparatus 420. The processor 120 is coupled to the physiological signal sensor 110, the compensation element 410, and the storage apparatus 420. Multiple code snippets are stored in the storage apparatus 420. The code snippets are executed by the processor 120 after being installed to execute a physiological signal recognition method. The code snippets may be composed into a system module 42. The system module 42 includes a noise variation computing module 421, a frequency domain conversion module 422, a noise reduction module 423, and an inverse frequency domain conversion module 424. Steps of the physiological signal recognition method are described below in conjunction with the system module 42.

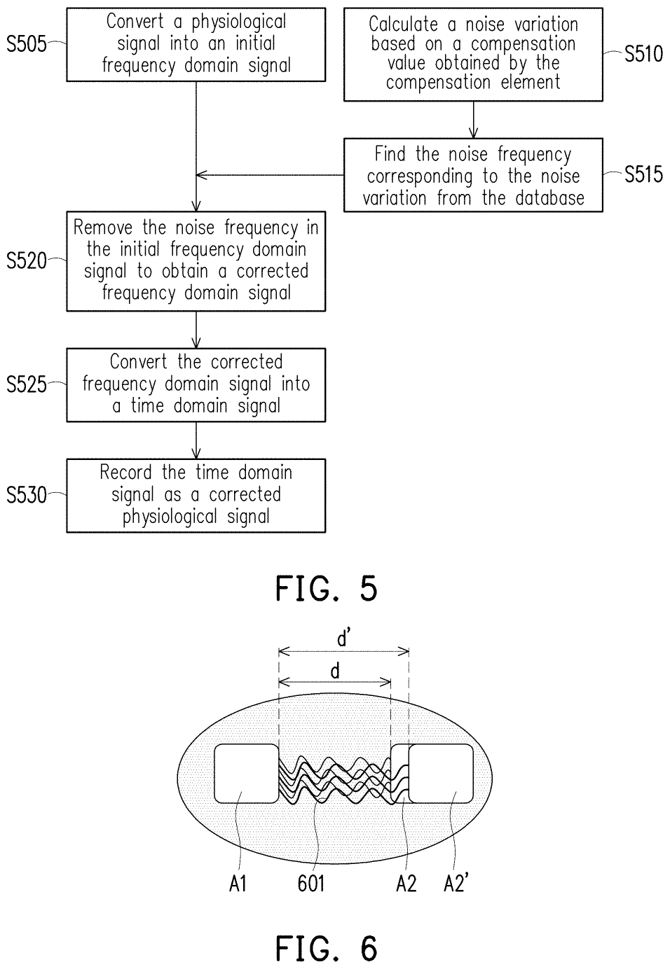

FIG. 5 is a flowchart of a physiological signal recognition method according to an embodiment of the disclosure. Please refer to FIG. 4 and FIG. 5. In Step S505, the frequency domain conversion module 422 converts a physiological signal into an initial frequency domain signal. For example, the frequency domain conversion module 422 adopts a Fourier transform algorithm to convert the physiological signal in the time domain into the frequency domain to obtain the initial frequency domain signal.

Next, in Step S510, the noise variation computing module 421 calculates a noise variation based on a compensation value obtained by the compensation element 410. The compensation element 410 is configured to measure a resistance between two electrodes in the physiological signal sensor 110 as the compensation value. The noise variation computing module 421 calculates the noise variation based on the compensation value.

Table 1 shows the lookup table of the noise variation. Different compensation values have corresponding noise variations, where xo is the compensation value (resistance value) measured when the two electrodes in the physiological signal sensor 110 are not stretched.

TABLE 1

| | | | | | | |
|---|---|---|---|---|---|---|
| Physiological signal | S₀ | S₁ | S₂ | S₃ | . . . | Sₙ |
| Compensation value (resistance value) | x₀ | x₁ | x₂ | x₃ | . . . | xₙ |
| Noise variation | D₀ = 0 | D₁ | D₂ | D₃ | . . . | Dₙ |

In Table 1, the initial setting of a noise variation Do when the two electrodes are not stretched is 0, and other noise variations D₁ to Dₙ are calculated based on the following equation (1).

$${D}_{i} = \sqrt{\frac{{\sum }_{i = 0}^{n} {( {x}_{i} - \overline{x} )}^{2}}{n - 1}} \qquad ( 1 )$$

where D₁ is the i-th noise variation, xᵢ is the i-th compensation value, and X̅ is the average value of compensation values. That is, every time a compensation value is obtained, the compensation value is filled in Table 1 for calculation.

In addition, a stretching distance between the two electrodes may also be measured by the compensation element 410 as the compensation value. FIG. 6 is a schematic diagram of sensing electrodes according to an embodiment of the disclosure. In this embodiment, a stretchable capacitor/resistor 601 is used as the compensation element 410. The stretchable capacitor/resistor 601 is disposed between electrodes A1 and A2. In addition, the electrode A2 after displacement is represented by an electrode A2′. The distance before stretching is d, and the distance after stretching is d′, so the stretching distance is d′-d.

For example, when the stretching distance is 1 mm, the noise variation is CV1; when the stretching distance is 2 mm, the noise variation is CV2, and so on. Alternatively, it may also be set such that when the stretching distance falls within a range of 0 to 1 mm, the noise variation is CV1; when the stretching distance falls within a range of 1 to 2 mm, the noise variation is CV2, and so on.

In addition, the compensation element 410 may also be implemented with multiple capacitors or gyroscopes, which may detect multi-directional stretching action patterns. For example, multiple capacitors are used to sense the stretching of the electrodes in multiple directions or a gyroscope is used to sense twisting and stretching deformation, so as to measure the stretching distance between the two electrodes.

In addition, the compensation element 410 may also be used to measure conductivity as the compensation value. That is, the compensation element 410 is used to sense skin perspiration to obtain the conductivity. After that, the processor 120 finds a noise frequency corresponding to the conductivity from a database.

Table 2 shows the correspondence between the conductivity and the frequency.

TABLE 2

Conductivity

| Frequency | 10% | 20% | . . . | 100% |
|---|---|---|---|---|
| 10 Hz | 1 db | 0 | . . . | 2 db |
| 20 Hz | 3 db | 0 | . . . | 0 |
| 30 Hz | 0 | 4 db | . . . | 5 db |
| . . . | . . . | . . . | . . . | . . . |
| . . . | . . . | . . . | . . . | . . . |

In terms of conductivity of 10%, if the compensation element 410 detects that the conductivity is 10%, it is found by looking up the table that there are amplitudes at frequencies of 10 Hz and 20 Hz, which are respectively 1 db and 3 db. Therefore, the frequencies of 10 Hz and 20 Hz are used as the noise frequency.

After obtaining the noise variation, the noise variation computing module 421 finds the noise frequency corresponding to the noise variation from the database in Step S515. That is, one or more noise frequencies corresponding to different noise variations may be established in the storage apparatus 420 in advance. After obtaining the noise variation, the corresponding noise frequency may be obtained by looking up the table.

After that, in Step S520, the noise reduction module 423 removes the noise frequency in the initial frequency domain signal to obtain a corrected frequency domain signal. Then, in Step S525, the inverse frequency domain conversion module 424 converts the corrected frequency domain signal into a time domain signal. In Step S530, the processor 120 records the time domain signal as a corrected physiological signal.

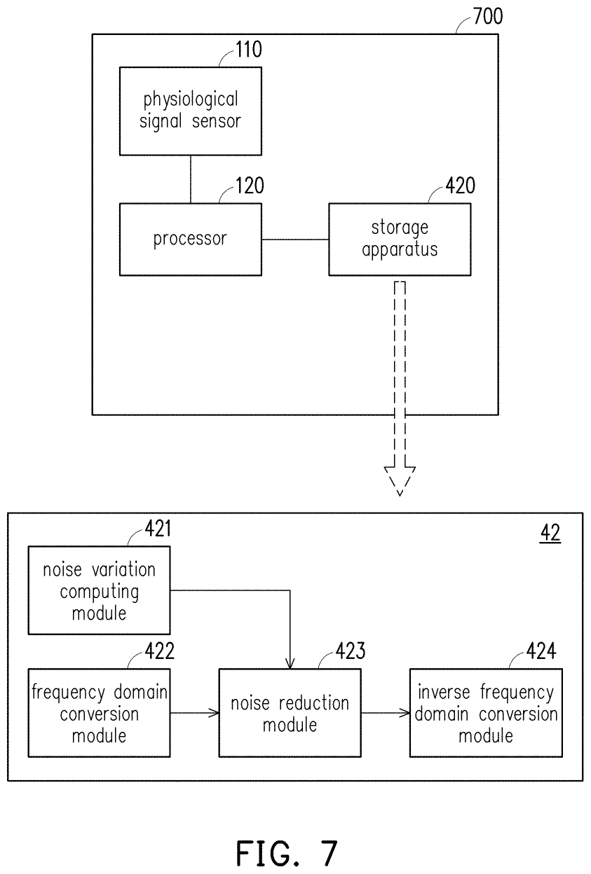

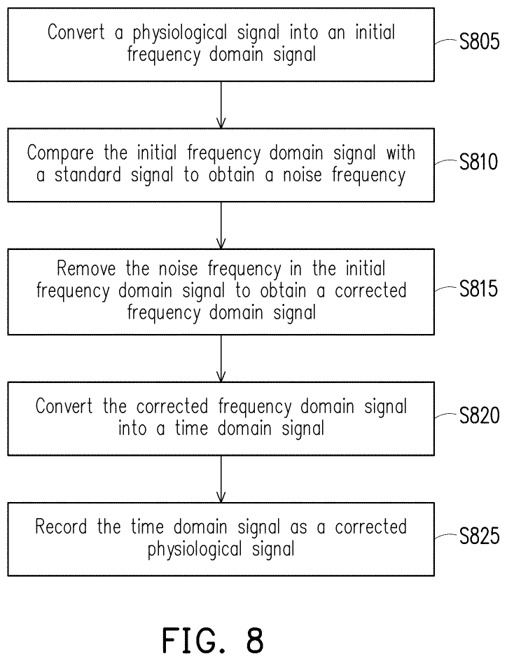

In other embodiments, the compensation element may not be used, and the noise frequency may be directly obtained based on a physiological signal and a standard signal. FIG. 7 is a block diagram of a physiological signal recognition apparatus according to an embodiment of the disclosure. FIG. 8 is a flowchart of a physiological signal recognition method according to an embodiment of the disclosure. In this embodiment, the difference between a physiological signal recognition apparatus 700 and a physiological signal recognition apparatus 400 is that the physiological signal recognition apparatus 700 does not have the compensation element 410.

In Step S805, the frequency domain conversion module 422 converts a physiological signal into an initial frequency domain signal. Next, in Step S810, the noise variation computing module 421 compares the initial frequency domain signal with a standard signal to obtain a noise frequency. Here, when starting to activate the physiological signal recognition apparatus 700, an initial setting is first performed to obtain an initial physiological signal that has not yet started to perform an action, and the initial physiological signal is converted into a time domain signal as the standard signal for subsequent comparison. For example, the standard signal is subtracted from the initial frequency domain signal to obtain the noise frequency.

After that, in Step S815, the noise reduction module 423 removes the noise frequency in the initial frequency domain signal to obtain a corrected frequency domain signal. Then, in Step S820, the inverse frequency domain conversion module 424 converts the corrected frequency domain signal into a time domain signal. In Step S825, the processor 120 records the time domain signal as a corrected physiological signal.

In addition, the physiological signal recognition methods shown in FIG. 5 and FIG. 8 may further execute the RMS algorithm on the corrected physiological signal after obtaining the corrected physiological signal to obtain a noise threshold, and adjust the corrected physiological signal based on the noise threshold to obtain an adjusted signal. In other words, the system module 200 and the system module 42 may be integrated.

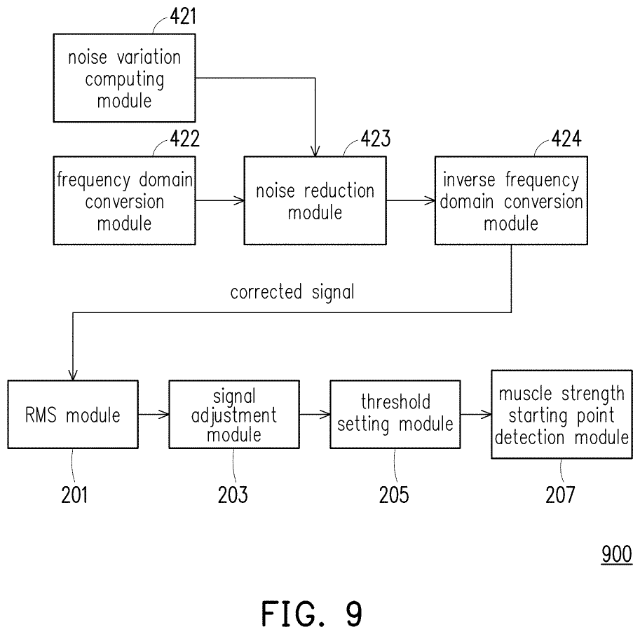

FIG. 9 is a block diagram of a system module according to an embodiment of the disclosure. A system module 900 of this embodiment is obtained by integrating the system module 200 and the system module 42. After a correction procedure is performed on the physiological signal through the noise variation computing module 421, the frequency domain conversion module 422, the noise reduction module 423, and the inverse frequency domain conversion module 424 to obtain the corrected physiological signal, the inverse frequency domain conversion module 424 transmits the corrected physiological signal to the RMS module 201. After that, the RMS module 201, the signal adjustment module 203, the threshold setting module 205, and the muscle strength starting point detection module 207 adjust the corrected physiological signal to detect a muscle strength starting point in an adjusted signal. For detailed description, please refer to the related descriptions of FIG. 2, FIG. 3A, and FIG. 3B.

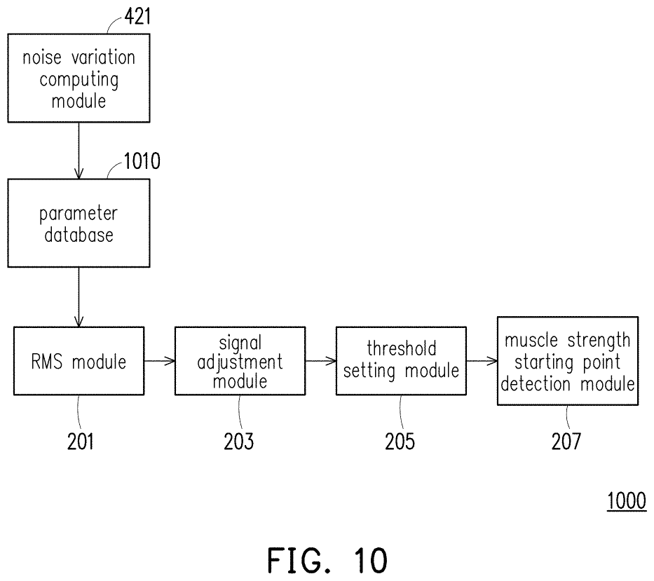

FIG. 10 is a block diagram of a system module according to an embodiment of the disclosure. In this embodiment, a system module 1000 includes a noise variation computing module 421, a parameter database 1010, an RMS module 201, a signal adjustment module 203, a threshold setting module 205, and a muscle strength starting point detection module 207. After obtaining a noise variation, the noise variation computing module 421 stores the noise variation in the parameter database 1010. The RMS module 201 queries the parameter database 1010 to obtain the noise variation, so as to change parameters used for setting a standard deviation value in the RMS algorithm.

The foregoing embodiments may be applied in scientific sports training, and may accurately analyze the starting sequence of each muscle to perform corresponding training adjustments. For example, the foregoing embodiments may be applied in sports training such as baseball, physical fitness, and golf training. The foregoing embodiments may also be applied in health care such as rehabilitation and long-term care, and may confirm whether a rehabilitation action is correct. The timing difference of antagonistic muscles is also an indicator of muscle and joint variation. The foregoing embodiments may also be applied in labor safety monitoring to analyze labor with long-term force exertion. For example, magnitudes of left and right muscle strengths, difference in muscle contraction time, excessive timing difference of antagonistic muscles of hands are detected as warning signals of the body for the reference of the employer.

Based on the above, the embodiments of the disclosure can detect noise in real time, thereby correcting the signal to improve dynamic accuracy and reduce signal distortion.

In summary, the disclosure corrects the signal by separating the noise from the main signal through the algorithm to improve dynamic accuracy and reduce signal distortion. Moreover, the use of the weight adjustments may reduce the amplitude of noise and maintain the amplitude of the main frequency. In addition, the starting signal threshold may be adjusted according to the action speed of the user to improve the recognition rate of the muscle strength starting point.

It will be apparent to those skilled in the art that various modifications and variations can be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. A physiological signal recognition apparatus, comprising:
a physiological signal sensor, sensing a physiological signal; and
a processor, coupled to the physiological signal sensor and configured to:
execute a root mean square algorithm on the physiological signal to obtain a noise threshold;
adjust the physiological signal based on the noise threshold to obtain an adjusted signal; and
detect a muscle strength starting point in the adjusted signal.

2. The physiological signal recognition apparatus according to claim 1, wherein the processor is configured to: multiply an amplitude in the physiological signal that is less than the noise threshold by a first weight value and multiply an amplitude in the physiological signal that is greater than or equal to the noise threshold by a second weight value to obtain the adjusted signal.

3. The physiological signal recognition apparatus according to claim 1, wherein the processor is configured to: set a starting signal threshold, and detect the muscle strength starting point in the adjusted signal based on the starting signal threshold.

4. The physiological signal recognition apparatus according to claim 3, wherein the processor is configured to: set the starting signal threshold according to an action speed.

5. The physiological signal recognition apparatus according to claim 1, wherein the processor is configured to: execute a correction procedure before executing the root mean square algorithm on the physiological signal to execute the root mean square algorithm on a corrected physiological signal after obtaining the corrected physiological signal, wherein
the correction procedure comprises:
converting the physiological signal into an initial frequency domain signal;
searching a database to obtain a noise frequency;
removing the noise frequency in the initial frequency domain signal to obtain a corrected frequency domain signal;
converting the corrected frequency domain signal into a time domain signal; and
recording the time domain signal as the corrected physiological signal.

6. The physiological signal recognition apparatus according to claim 5, further comprising:
a compensation element, coupled to the processor and configured to obtain a compensation value, wherein
the processor is configured to: calculate a noise variation based on the compensation value, and find the noise frequency corresponding to the noise variation from the database.

7. The physiological signal recognition apparatus according to claim 6, wherein the compensation element is configured to measure a stretching distance between two electrodes of the physiological signal sensor as the compensation value; and
the processor is configured to: obtain a resistance value based on the stretching distance, and calculate the noise variation based on the resistance value.

8. The physiological signal recognition apparatus according to claim 6, wherein the compensation element is configured to measure a conductivity as the compensation value; and
the processor is configured to: find the noise frequency corresponding to the conductivity from the database.

9. The physiological signal recognition apparatus according to claim 5, wherein the processor is configured to: search the database and compare the initial frequency domain signal with a standard signal to obtain the noise frequency.

10. The physiological signal recognition apparatus according to claim 1, wherein the physiological signal is an electromyography signal.

11. A physiological signal recognition method, comprising:
converting a physiological signal into an initial frequency domain signal;
calculating a noise variation based on a compensation value obtained by a compensation element;
finding a noise frequency corresponding to the noise variation from a database;
removing the noise frequency in the initial frequency domain signal to obtain a corrected frequency domain signal;
converting the corrected frequency domain signal into a time domain signal; and
recording the time domain signal as a corrected physiological signal.

12. The physiological signal recognition method according to claim 11, wherein the step of calculating the noise variation based on the compensation value obtained by the compensation element comprises:
measuring a stretching distance between two electrodes of the physiological signal sensor through the compensation element as the compensation value; and
obtaining a resistance value based on the stretching distance, and calculating the noise variation based on the resistance value.

13. The physiological signal recognition method according to claim 11, wherein the step of calculating the noise variation based on the compensation value obtained by the compensation element comprises:
measuring a conductivity through the compensation element as the compensation value; and
finding the noise frequency corresponding to the conductivity from the database.

14. The physiological signal recognition method according to claim 11, further comprising:
executing a root mean square algorithm on the corrected physiological signal to obtain a noise threshold; and
adjusting the corrected physiological signal based on the noise threshold to obtain an adjusted signal.

15. The physiological signal recognition method according to claim 14, wherein the step of adjusting the corrected physiological signal based on the noise threshold to obtain the adjusted signal comprises:
multiplying an amplitude in the corrected physiological signal that is less than the noise threshold by a first weight value and multiplying an amplitude in the corrected physiological signal that is greater than or equal to the noise threshold by a second weight value to obtain the adjusted signal.

16. The physiological signal recognition method according to claim 14, wherein after the step of adjusting the corrected physiological signal based on the noise threshold to obtain the adjusted signal, the physiological signal recognition method further comprises:
setting a starting signal threshold according to an action speed, and detecting a muscle strength starting point in the adjusted signal based on the starting signal threshold.

17. A physiological signal recognition method, comprising:
converting a physiological signal into an initial frequency domain signal;
comparing the initial frequency domain signal with a standard signal to obtain a noise frequency;
removing the noise frequency in the initial frequency domain signal to obtain a corrected frequency domain signal;
converting the corrected frequency domain signal into a time domain signal; and
recording the time domain signal as a corrected physiological signal.

18. The physiological signal recognition method according to claim 17, further comprising:
executing a root mean square algorithm on the corrected physiological signal to obtain a noise threshold; and
adjusting the corrected physiological signal based on the noise threshold to obtain an adjusted signal.

19. The physiological signal recognition method according to claim 18, wherein the step of adjusting the corrected physiological signal based on the noise threshold to obtain the adjusted signal comprises:
multiplying an amplitude in the corrected physiological signal that is less than the noise threshold by a first weight value and multiplying an amplitude in the corrected physiological signal that is greater than or equal to the noise threshold by a second weight value to obtain the adjusted signal.

20. The physiological signal recognition method according to claim 18, wherein after the step of adjusting the corrected physiological signal based on the noise threshold to obtain the adjusted signal, the physiological signal recognition method further comprises:
setting a starting signal threshold according to an action speed, and detecting a muscle strength starting point in the adjusted signal based on the starting signal threshold.
